(19) United States
(12) Patent Application Publication
(10) Pub. No.: US 2006/0286116 A1
(43) Pub. Date: Dec. 21, 2006

(54) Methods and compositions for treating B cell cancer

(75) Inventors: James G. McArthur, Concord, MA (US); Linda Liang, Somerville, MA (US)

Correspondence Address: WILMER CUTLER PICKERING HALE AND DORR LLP, 60 STATE STREET, BOSTON, MA, 02109, US

(73) Assignee: Morningside Venture Investments Limited

(21) Appl. No.: 11/432,235

(22) Filed: May 11, 2006

Related U.S. Application Data
(63) Continuation of application No. PCT/US04/37705, filed on Nov. 12, 2004.
(60) Provisional application No. 60/519,182, filed on Nov. 12, 2003.

(51) Int. Cl. A61K 39/00; G01N 33/574

(52) U.S. Cl. 424/185.1; 435/7.23

(57) ABSTRACT

Disclosed are methods for killing or retarding the growth of a B cell cancer cell, by contacting the cell with a FRIL family member molecule. Also disclosed are methods for determining if a B cell cancer is sensitive to a FRIL family member molecule, or if a B cell cancer patient will benefit from treatment with a FRIL family member molecule, by contacting a B cell cancer cell with the FRIL family member molecule and determining if growth of the cell is retarded. In addition, methods for treating a B cell cancer patient are disclosed, as well as compositions comprising a FRIL family member molecule and a chemotherapeutic, radiotherapeutic, or agent that selectively kills B cells. Also disclosed are methods for locating a B cell cancer in a patient by administering a detectably labeled FRIL family member molecule to the patient and detecting the label.

### BACKGROUND OF THE INVENTION

1. Field of the Invention

The invention relates to B cell cancers and the treatment thereof.

2. Background

There are over 1 million new cancer cases diagnosed each year in the United States and, while there are many therapies and treatment regimens for cancer, over 500,000 people in the United States die from cancer each year. According to the National Cancer Institute, lymphomas are the fifth most common malignancy diagnosed in the United States with approximately 60,000 new cases and the sixth leading cause of cancer-related deaths. Among lymphomas, Non-Hodgkin's Lymphomas caused by malignant (cancerous) B lymphocytes represent the majority (about 85% in the US) of lymphoma cases.

Another type of cancer caused by malignant B lymphocytes, namely B cell Acute Lymphocytic Leukemia (B-ALL), has a significant societal impact since it predominantly afflicts children under the age of 15 with approximately 2,000 new cases annually.

While 5-year survivals for B-NHL and B-ALL are 50% to 65%, these diseases are rarely completely cured. Given the large number of deaths from B cell cancer, and the difficulties in treating this disease with known methods, there is a need to discover new methods and compositions for treating B cell cancer.

### SUMMARY OF THE INVENTION

The invention provides methods and compositions for treating B cell cancer and for retarding the growth of and/or killing cancerous B cells.

Accordingly, in a first aspect, the invention provides a method for killing a cell of a B cell cancer, comprising contacting the cell with a FRIL family member molecule.

In a further aspect, the invention provides a method for retarding the growth of a cell of a B cell cancer, comprising contacting the cell with a FRIL family member molecule, wherein the FRIL family member molecule binds to a molecule on the surface of the cell that is not a normally glycosylated FLT3 receptor. In certain embodiments, the growth of the cell is abrogated. In particular embodiments, the cell is killed.

In certain embodiments, the cell is cultured in vitro. In some embodiments, the cell is in a patient (e.g., a human patient). In some embodiments, the FRIL family member molecule is administered to the patient.

In another aspect, the invention features a method for treating a patient (e.g., a human patient) suffering from a B cell cancer, comprising administering to the patient a composition comprising a therapeutically effective amount of a FRIL family member and a pharmaceutically acceptable carrier.

In a further aspect, the invention provides a composition comprising a FRIL family member molecule and a chemotherapeutic, a radiotherapeutic, or an agent that selectively kills B cells.

In a further aspect, the invention features a method for determining if a patient (e.g., a human patient) suffering from a B cell cancer will benefit from treatment with a FRIL family member molecule, comprising contacting a cell from the B cell cancer with a FRIL family member molecule and determining the growth rate of the cell, wherein retardation of growth of the cell contacted with the FRIL family member molecule as compared to a cell from the B cell cancer not contacted with the FRIL family member molecule indicates that patient will benefit from treatment with the FRIL family member molecule.

In yet another aspect, the invention features a method for determining if a B cell cancer is sensitive to a FRIL family member molecule comprising contacting a cell from the B cell cancer with the FRIL family member molecule and determining the growth rate of the cell, wherein retardation of growth of the cell contacted with the FRIL family member molecule as compared to a cell from the B cell cancer not contacted with the FRIL family member molecule indicates that the B cell cancer is sensitive to the FRIL family member molecule.

In yet another aspect, the invention provides a method for locating a cell of a B cell cancer in a patient (e.g., a human patient) comprising administering a detectably labeled FRIL family member molecule to the patient and detecting the label. In certain embodiments, the detectable label is radioactive, chromophoric, or fluorogenic. In some embodiments, the detectable label is an agent used in extra-corporal imaging of tissues

In various embodiments of all of the aspects of the invention, the growth of a cell of the B cell cancer is retarded. In certain embodiments, the growth of a cell of the B cell cancer is abrogated. In particular embodiments, a cell of the B cell cancer is killed.

In some embodiments of all of the aspects of the invention, the B cell cancer is a B cell Non-Hodgkin Lymphoma. In certain embodiments, the B cell Non-Hodgkin Lymphoma is a small lymphocytic lymphoma (SLL), a mantle cell lymphoma, a Burkitt's lymphoma, a follicle centre cell lymphoma, a follicular lymphoma, a Burkitt-like lymphoma, a marginal zone B-cell lymphoma (MZBCL), a nodal marginal zone B cell lymphoma, an extra-nodal marginal zone B cell lymphoma, a splenic marginal zone B cell lymphoma, a lymphoplasmacytic lymphoma, or a diffuse large B cell lymphoma.

In certain embodiments of all aspects of the invention, the B cell cancer is a B cell acute lymphocytic leukemia (B-ALL), a precursor B cell acute lymphocytic leukemia (B-ALL), a B cell chronic lymphocytic leukemia (B-CLL), a precursor B-lymphoblastic leukaemia, a precursor B-lymphoblastic lymphoma, a small lymphocytic lymphoma, a B cell prolymphocytic leukemia, an undifferentiated B cell lymphoma, a hairy cell leukemia, a mediastinal large B-cell lymphoma, a plasma cell myeloma, a plasmacytoma, a primary effusive lymphoma, a Burkitt's cell leukemia, or a B cell diffuse mixed lymphoma.

In certain embodiments of all of the aspects of the invention, the FRIL family member molecule is purified. The FRIL family member molecule may be Dl-FRIL, Yam-FRIL, or Pv-FRIL.

### BRIEF DESCRIPTION OF THE DRAWINGS

FIG. 1 is a schematic representation of a line graph showing the percentage of normal human B cells (white squares), and two B cell tumor lines, CCRF-SB (black circles) and JM-1 (black triangles), which were stained with the indicated concentrations of biotinylated FRIL followed by the secondary, streptavidin-PE.

FIG. 2 is a schematic representation of a line graph showing the number of viable cells (as determined by their conversion of XTT to formazan) following contact with DI-FRIL, a non-limiting FRIL family member molecule of the invention. The cell lines tested were: KG1a (white squares); JM1 (white circles); SR (white triangles); RL (black rectangles); Raji (black circles) MC116 (black squares); HT (black triangles); and CCRF (black diamonds).

FIG. 3 is a schematic representation of a line graph showing the killing of B cell lymphoma cells following incubation of normal B cells (circles) or MC116 lymphoma cells (squares) with 0.2 μg/ml (white symbols) or 10 μg/ml (black symbols) of Dl-FRIL, a non-limiting FRIL family member molecule of the invention.

FIG. 4 is a schematic representation of a line graph showing the killing of CCRF-SB cells (a Burkitt's B-ALL cell line) following incubation the indicated concentrations of two different FRIL family members; DlFRIL (black circles) and PvFRIL (black triangles).

### DETAILED DESCRIPTION OF THE PREFERRED EMBODIMENTS

The invention is based on the discovery that FRIL family member molecules bind to and retard the growth of and/or kill cancerous B cells. Thus, the invention provides methods for killing or retarding the growth of cancerous B cell (i.e., cells of a B cell cancer). The invention also provides methods for treating patients suffering from (or suspected of suffering from) B cell cancer.

All of the patents and publications cited herein reflect the knowledge in the art and are hereby incorporated by reference in entirety to the same extent as if each were specifically stated to be incorporated by reference. Any inconsistency between these patents and publications and the present disclosure shall be resolved in favor of the present disclosure.

Previous studies have found that individual members of the FRIL family of lectins are able to preserve progenitor cells (see, e.g., Moore et al., Biochim. Biophys. Acta 25027: 1-9, 2000). By “preserve” means that a FRIL family member molecule retains (i.e., preserves) progenitor cells in an undifferentiated state, which can be determined using the art-known assays (see, e.g., the assays described in Colucci et al., PCT Application No. PCT/US99/31307 (PCT Publication No. WO01/49851)). A FRIL family member molecule (or simply “a FRIL family member”) preserves progenitor cells by binding to a normally glycosylated FLT3 receptor on the progenitor cell (see, e.g., Moore et al., Biochim. Biophys. Acta 25027: 1-9, 2000). The FLT3 receptor is the receptor for the FLT3 ligand, a hematopoietic growth factor that plays a key role in growth of primitive hematopoietic cells. The FLT3 receptor is normally glycosylated on normal (i.e., non-cancerous cells) and aberrantly glycosylated on cancerous cells.

A progenitor cell preserved by contact with a FRIL family member molecule does not divide as rapidly as a progenitor cell not contacted with the FRIL family member molecule. Eventually, of course, as the FRIL family member molecule in the culture dish (or in the treated animal) is depleted, the contacted progenitor cell “wakes up” and divides normally.

As described herein, FRIL family member molecules have been discovered to bind to a second molecule, one that expressed on cancerous B cells. B cells (also called B lymphocytes) are those white blood cells which express and/or secrete antibodies (also called immunoglobulins). Non-limiting cell surface molecules expressed by B cells include CD19, CD20, CD22, CD72, CD79a, CD79β, CD121b, and CD138. Although the exact cell surface molecule on a cancerous B cell to which the FRIL family member binds (also called the ligand to which the FRIL family member binds) has not yet been determined, it is not the FLT3 receptor, since neither normal nor cancerous B cells express the FLT3 receptor.

Moreover, as described herein, FRIL family member molecules have been discovered to have an unexpected ability to retard the growth of or kill cancerous B cells. This ability to retard the growth of or kill cancerous B cells must be mediated through a FRIL family member's binding to the as-yet-unknown cell surface molecule on cancerous B cells, since FRIL family members bind to the FLT3 receptor on progenitor cells without killing them. Thus, a patient suffering from B cell cancer who is treated by administration of a FRIL family member molecule will have his cancerous B cells killed while his progenitor cells, albeit temporarily preserved by contact with the FRIL family member molecule, are unharmed and return to normal as soon as the FRIL family member molecule in the patient is depleted.

Accordingly, in one aspect, the invention provides a method for killing a cell of a B cell cancer, comprising contacting the cell with a FRIL family member molecule. In some embodiments, the FRIL family member contacts the cell by binding to a molecule on the surface of the cell, where the cell surface molecule is not a normally glycosylated FLT3 receptor.

In a further aspect, the invention provides a method for retarding the growth of a cell of a B cell cancer, comprising contacting the cell with a FRIL family member molecule, wherein the FRIL family member molecule binds to a molecule on the surface of the cell that is not a normally glycosylated FLT3 receptor.

FRIL family members (or FRIL family member molecules) are mannose/glucose-specific legume lectins (i.e., proteins that binds sugar residues with high affinity). (See Moore et al., Biochim. Biophys Acta 25027: 1-9, 2000; Colucci et al., Proc. Natl. Acad. Sci. USA 96: 646-650, 1999; Mo et al., Glycobiology 9: 173-179, 1999; and Hamelryck et al., J. Molec. Biol. 299: 875-883, 2000). FRIL family member molecule may be isolated from members of the tribe Phaseoleae including, without limitation, Phaseolus vulgaris, Dolichos lab lab, Sphenostylis stenocarpa, Cicer arietinum, Sphenostylus stenocarpa, Phaseolus acutifolius, Phaseolus lunatus, Vigna sinensis, or Voandzeia subterranea. In certain embodiments, the FRIL family member molecule that is isolated from a hyacinth bean (i.e., Dolichos lab lab) has an amino acid sequence which comprises the following eight amino acid sequence: TNNVLQXT (SEQ ID NO:11; where X is any amino acid). A FRIL family member of the invention is, in some embodiments, encoded by a nucleic acid molecule comprising or consisting of the sequence of SEQ ID NO:1, SEQ ID NO:3, SEQ ID NO:5, SEQ ID NO:7, SEQ ID NO:12, SEQ ID NO:15, or SEQ ID NO: 18. In some embodiments, the FRIL family member molecule of the invention has an amino acid sequence comprising or consisting of the sequence of SEQ ID NO:2, SEQ ID NO:4, SEQ ID NO:6, SEQ ID NO:8, SEQ ID NO:9, SEQ ID NO:10, SEQ ID NO:11, SEQ ID NO: 13, SEQ ID NO: 14, SEQ ID NO: 16, SEQ ID NO: 17, SEQ ID NO: 19, or SEQ ID NO: 20.

Other molecules falling into the definition of a FRIL family member molecule (e.g., mutants or fusion proteins), recombinant FRIL family member molecules, and methods for making and purifying such FRIL family member molecules (and methods for purifying nucleic acid molecules encoding such FRIL family member molecules) are described in U.S. Pat. No. 6,084,060; U.S. Pat. No. 6,280,724; U.S. Pat. No. 6,310,195; Colucci et al., PCT Application No. PCT/US99/31307 (PCT Publication No. WO 01/49851); Moore, J. G., PCT Application No. PCT/US02/05763 (PCT Publication No. WO02/067973); Colucci et al., PCT Application No. PCT/US98/13046 (PCT Publication No. WO98/59038), and Moore, J. G., PCT Application No. PCT/US02/21355 (PCT Publication No. WO03/004616), the entire disclosures of all of which are hereby incorporated by reference.

In accordance with the invention, each FRIL family member molecule has at least about 45% amino acid sequence identity with the amino acid sequence of another member of the FRIL family, or at least about 55% amino acid sequence identity, or at least about 65% amino acid sequence identity, or at least about 75% amino acid sequence identity, or at least about 85% amino acid sequence identity with the amino acid sequence of another member of the FRIL family. In certain embodiments, each FRIL family member molecule has at least about 95% identity with the amino acid sequence of another member of the FRIL family (e.g., SEQ ID NO:2, SEQ ID NO:4, SEQ ID NO:6, SEQ ID NO:8, SEQ ID NO:9, or SEQ ID NO:10). Amino acid sequence identity and nucleic acid sequence identity between two proteins or two nucleic acid molecules can be measured according to standard methods (see, e.g., Pearson and Lipman, Proc. Natl. Acad. Sci. USA 85: 2444-2448, 1988; George, D. G. et al., in Macromolecular Sequencing and Synthesis, Selected Methods and Applications, pps. 127-149, Alan R. Liss, Inc. 1988; Feng and Doolittle, Journal of Molecular Evolution 25: 351-360, 1987; and Higgins and Sharp, CABIOS 5: 151-153, 1989; and the various BLAST programs of the National Center for Biotechnology, National Library of Medicine, Bethesda, Md.).

The terms, “cancerous B cell” and “cell of a B cell cancer” are used interchangeably herein to refer to a B cell that is cancerous. By “cancerous cell” or “cancer cell” is meant a cell that shows aberrant cell growth, such as increased cell growth. A cancerous cell may be a hyperplastic cell, a cell that shows a lack of contact inhibition of growth in vitro, a tumor cell that is incapable of metastasis in vivo, or a metastatic cell that is capable of metastasis in vivo. In certain embodiments, the cancerous B cell of the invention is capable of metastasis in vivo.

In some embodiments of the various aspects of the invention, the B cell cancer is a B cell Non-Hodgkin Lymphoma. Table I, which is taken from Antonio Cuneo “Classification of B-cell non-Hodgkin's lymphomas (NHL): cytogenetic entities, immunopheneotype and clinical features” Atlas Genet. Cytogenet. Oncol. Haematol. February 2000, describes numerous types of B cell Non-Hodgkin Lymphomas.

TABLE I
Types of B cell Non-Hodgkin's Lymphomas

| Histologic subset | | | |
|---|---|---|---|
| and | | Cytogenetic entitiy and corresponding clinical | |
| Immunophenotype | Putative cell of origin | features | |
| Small lymphocytic | CD5+ virgin B-cell | del(6)(q21-23) (20-30% | Indolent disease; |
| lymphoma (SLL) | with germline IgV | of the cases) | leukemic involvement by |
| Pan-B+; CD5+; | genes¹ | | lymphoid cells, including |
| CD23+; CD10−; | | | prolymphocytes and/or |
| sIgM+ faint | | | paraimmunoblasts |
| | | | Splenomegaly |
| Lymphoplasmacytic | Peripheral B- | t(9; 14)(p13; q32) | Indolent low-grade |
| lymphoma | lymphocyte | PAX5/IgH (50% of | disease, with possible |
| Pan-B+; CD5−; | transforming into | cases) | clinical and/or histologic |
| CD10−; cyIgM+ | plasma cell with | | progression |
| | mutated IgV genes | | |
| | and ongoing | | |
| | mutations | | |
| Follicle centre cell | Centrocytes/ | t(14; 18)(q32; q21)/ | Indolent. Advanced |
| lymphoma | centroblasts of | BCL2 Rearr (70-80% | stages predominate. |
| Pan-B+; CD10+/−; | germinal centre | of cases) | Conflicting data as to |
| CD5−; sIg+ | origin with somatic | | the prognostic significance |
| | hypermutation of the | | of the t(14; 18)/BCL2Rearr |
| | IgV genes and | | |
| | ongoing mutations | | |
| | (antigen driven | | |
| | stimulation) | | |
| Diffuse large cell | Large transformed B- | t(14; 18) and p53 | Usually aggressive |
| lymphoma | cells harbouring | mutations (20% of | Immunoblastic |
| CD19+; CD22+; | somatic | the cases) | lymphoma (Kiel |
| CD10−/+; SIg+ | hypermutation of the | t(3; V)(q27; V)/ | classification) do worse |
| | Ig genes | BCL6 Rearr (6-30% | than centroblastic |
| | (ongoing mutations in | of cases²) | lymphomas |
| | some cases) | t(8; 14)(q24; q32) or | No convincing |
| | | variants c-MYC | demonstration that any |
| | | Rearr (7-10% of | “primary” cytogenetic/ |
| | | cases) | molecular defect has |
| | | | prognostic significance; |
| | | | complex karyotype |
| | | | confers a shorter survival |
| Burkitt's lymphoma | Peripheral B-cells | t(8; 14)(q24; q32) or | Extremely aggressive |
| Pan-B+; TdT−; | that have encountered | variants | disease |
| CD10+; CD5−; | the antigen and | | Specific treatment |
| sIgM+ | harbours somatic | | mandatory |
| | hypermutation of the | | |
| | Ig genes | | |
| Burkitt-like | Peripheral B-cells | t(8; 14) or variants | Aggressive disease |
| lymphoma Pan-B+; | that have encountered | (25% of cases) | Cases with dual 8; 14 |
| TdT−; CD10−/+ | the antigen | t(8; 14) + t(14; 18) | and 14; 18 translocations |
| CD5−; sIg+ | | (30% of cases) | have a worse outcome |
| Mantle cell | CD5+ B-cells of the | t(11; 14)(q13; q32)/ | Advanced stages |
| lymphoma | follicle mantle having | BCL1 Rearr (50-90%) | predominate |
| Pan-B+; CD5+; | germline IgV gene | | Response to |
| CD23−; CD10−/+; | sequences | | chemotherapy often |
| sIgM+ bright | | | unsatisfactory |
| | | | Short survival |
| | | | Complex karyotype |
| | | | carries an unfavourable |
| | | | prognostic significance |
| Marginal zone B-cell | Marginal zone | t(11; 18)(q21; q21)/ | Extra-nodal low-grade |
| lymphoma | lymphocytes | PI2/MLT fusion | MALT lymphoma; |
| (MZBCL) | harbouring | (30-50% of the | indolent disease |
| pan-B+; CD5−/+; | hypermutated IgV | low-grade MALT) | |
| CD10−; CD23−; | genes | t(1; 14)(p21; q32) | Extra-nodal MALT |
| CD11c+/−; cyIg+ | | | lymphoma |
| (40% of the cells), | | del(7)(q22−31) | Splenic MZBCL |
| sIgM+ bright; sIgD−) | | (40% of the cases) | |
| | | +3/+3q | Nodal, extra-nodal and |
| | | (30-70% of the | splenic MZBCL |
| | | cases) | |

+: positive in more than 90% of the cases;
+/−: positive in more than 50% of the cases;
−/+: positive in less than 50% of cases;
−: positive in <10% of the cases;
pan-B markers include CD19; CD20; CD79a;
R = rearranged;
sIg: surface immunoglobulins;
cyIg: cytoplasmic Ig;
IgV genes: genes encoding for the variable portion of the Ig.

Additional B cell Non-Hodgkin's Lymphomas include the mediastinal large B-cell lymphoma, lymphoblastic B cell lymphoma, Waldenstrom's macroglobulinaemia, and follicular lymphoma. Thus, in certain embodiments, the B cell Non-Hodgkin's Lymphoma is small lymphocytic lymphoma (SLL), a mantle cell lymphoma, a Burkitt's lymphoma, a follicle centre cell lymphoma, a follicular lymphoma, a Burkitt-like lymphoma, a marginal zone B-cell lymphoma (MZBCL), a nodal marginal zone B cell lymphoma, an extra-nodal marginal zone B cell lymphoma, a splenic marginal zone B cell lymphoma, a lymphoplasmacytic lymphoma, or a diffuse large B cell lymphoma.

Of course, a FRIL family member of the invention is able to retard the growth of or kill cancerous B cells that are not non-Hodgkin Lymphoma cells. Thus, in certain embodiments of all aspects of the invention, the B cell cancer is a B cell acute lymphocytic leukemia (B-ALL), a precursor B cell acute lymphocytic leukemia (B-ALL), a B cell chronic lymphocytic leukemia (B-CLL), a precursor B-lymphoblastic leukaemia, a precursor B-lymphoblastic lymphoma, a small lymphocytic lymphoma, a B cell prolymphocytic leukemia, an undifferentiated B cell lymphoma, a hairy cell leukemia, a mediastinal large B-cell lymphoma, a plasma cell myeloma, a plasmacytoma, a primary effusive lymphoma, a Burkitt's cell leukemia, or a B cell diffuse mixed lymphoma.

As used herein, by “retarding the growth” means that contact with a FRIL family member slows the growth of a cancerous B cell. This growth retardation can be assessed by comparing the growth rate of a cancerous B cell contacted with a FRIL family member molecule to a cancerous B cell not contacted by that FRIL family member molecule. In some embodiments, the retardation causes a complete abrogation in growth (i.e., the cell contacted by the FRIL no longer divides). In some embodiments, the retardation causes death of the cell. Methods for determining cell growth are known in the art and include, without limitation, counting the cells or determining how much of a nutrient (e.g., ³H-thymidine) is taken up by the cells. These and other methods for determining cell growth are described in, e.g., Ausubel et al., Current Protocols in Molecular Biology, John Wiley & Sons, New York, N.Y., 1999, which is updated periodically.

As described in the Examples below, normal B cells are not FRIL sensitive (i.e., are FRIL insensitive), while most cancerous B cells are FRIL sensitive. As used herein, by “FRIL sensitive” is meant that a cancerous B cell is growth retarded by and/or killed by contact with a FRIL family member molecule. A FRIL family member molecule of the invention binds to normal B cells with low affinity, and binds to cancerous B cells with high affinity. Only those cancerous B cells to which a FRIL family member binds with high affinity are FRIL sensitive.

Without wishing to be bound to a particular theory, normal B cells which are not sensitive to FRIL may express the same FRIL family member ligand (i.e., the molecule on the cell surface of these cells to which the FRIL family member binds) as that expressed by cancerous B cells which are FRIL sensitive, but the cancerous B cells which are FRIL sensitive may express more of that ligand, while the FRIL insensitive normal B cells may express lower quantities of that ligand. Thus, the high affinity to FRIL observed in FRIL sensitive cancerous B cells may be due to the increased number of FRIL ligand that these cells express on their cell surface. Another possibility is that the FRIL family member ligand on FRIL sensitive cancerous B cells has undergone a different secondary modification (e.g., glycosylation pattern) than that ligand on FRIL insensitive normal B cells, such that the ligand on the FRIL sensitive cancerous B cells is able to bind a FRIL family member with higher affinity than the ligand expressed on FRIL insensitive normal B cells.

Yet another non-limiting possibility for explaining why cancerous B cells are FRIL sensitive while normal B cells are not is that intracellular biochemical and/or genetic changes that are a consequence (or cause) of the malignant phenotype of the B cell malignancies may render these cells more (or less) sensitive to the biochemical signals ensuing from FRIL binding to its ligand on these cells.

Moreover, although, as described in the Examples below, some cancerous B cells appear to be FRIL-insensitive, without wishing to be bound to a particular theory, it may be that these cells, due to their long propagation in vitro, have reduced the expression of the FRIL family member ligand (i.e., the molecule on the cell surface of these cells to which the FRIL family member binds), or have changed the secondary modification of the FRIL family member ligand. It is contemplated that a cancerous B cells arising in a patient (i.e., in vivo) will be FRIL sensitive.

FRIL binds with an EC₅₀ (i.e., the concentration of free FRIL required to elute 50% of the receptor-bound FRIL) of 0.5 to 08 μg/ml to FRIL sensitive cancerous B cells and an EC₅₀ of 2 to 4 μg/ml to normal human B cells. Similarly, greater than 5-fold more FRIL is required to achieve equivalent killing of normal human B cells as FRIL sensitive B-lymphomas. While B cell malignancies derived from mature B cell are recognized by FRIL, as described below, JM1, a pre-B cell leukemia, is not. While not wishing to be bound by any particular theory, this observation suggests that the B cell antigen recognized by FRIL may be expressed later in B cell differentiation.

In certain embodiments of the invention, the cancerous B cell is in a patient, such as a human patient. Other non-limiting patients of the invention include non-human primates, laboratory animals (e.g., mice, rats, rabbits, hamsters, frogs, and fish), livestock animals (e.g., horses, sheep, cows, pigs, goats, ostriches, chickens, turkeys, ducks, geese, elephants, and llamas), and pets (e.g., cats and dogs).

In certain embodiments of the invention, the cancerous B cells that are growth retarded by or killed by contact with a FRIL family member molecule are cultured in vitro. For example, the cancerous B cells may be from a similar type of cancer as that of a patient for whom treatment is sought (e.g., the cancerous B cell is a Burkitt's lymphoma and the patient is suffering from Burkitt's lymphoma). In this example, the cultured cancerous B cells may be used to predict what dosage of a FRIL family member useful for growth retarding and/or killing the cells. Different dosages of a FRIL family member can be added to the media in which the cells are cultured. The FRIL family member thus contacts the cells cultured in the media. The dosage of the FRIL family member that is found to growth retard and/or kill the cultured cells can then be used to treat a patient suffering from that type of cancer.

In another non-limiting example, the cancerous B cells may be removed from a patient suffering from a B cell cancer. In this example, the cancerous B cells are cultured in vitro and treated with different dosages of a FRIL family member molecule, in order to determine which dosage is most efficacious at killing or retarding the growth of the cancerous B cells. This dosage can then be used to treat the patient.

In some embodiments of the invention, the cancerous B cell is in a patient, and the cell is contacted with a FRIL family member molecule by administering the FRIL family member molecule to the patient. Administration routes and methods of a FRIL family member to a patient are described below. By administering a FRIL family member to a patient, contact of the cancerous B cell by the FRIL family member occurs inside the patient's body.

In yet another aspect, the invention features a method for determining if a B cell cancer is sensitive to a FRIL family member molecule comprising contacting a cell from the B cell cancer with the FRIL family member molecule and determining the growth rate of the cell, wherein retardation of growth of the cell contacted with the FRIL family member molecule as compared to a cell from the B cell cancer not contacted with the FRIL family member molecule indicates that the B cell cancer is sensitive to the FRIL family member molecule.

In accordance with the invention, a candidate cancerous B cell may be cultured in vitro. This cancerous B cell may be one taken from a biopsy of a patient suffering from a B cell cancer, or this cancerous B cell may be from an established tumor line (e.g., Raji or MC116). The cells may then be treated with no treatment, a control lectin, or different amounts of a FRIL family member molecule. Control lectins may be any lectin that is not a FRIL family member, and include, without limitation, phytohemagglutinin (PHA) (from kidney beans (i.e., Phaseolus vulgaris)), Concanavalin A (conA) (from jack beans) and WGA (from wheatgerm (i.e., Triticum aestivum)). Treatment may be made by simply adding the lectin to the cells' culture media, where the added lectin will make contact with the cultured cells. The treated cells are returned to the incubator for a specified time (e.g., 30 minutes, an hour, a day, or a week), and then are counted (e.g., using a hemacytometer, a Coulter cell counter, or a Guava PCA apparatus). If there are fewer cells in the culture dishes treated with the FRIL family member molecule as compared to the culture dishes that received no treatment or that received the control lectin, then the cancerous B cell is identified as being sensitive to the FRIL family member molecule. In some embodiments, all of the cultured cells are killed by the added FRIL family member molecule.

In another aspect, the invention features a method for treating a patient suffering from a B cell cancer, comprising administering to the patient a composition comprising a therapeutically effective amount of a FRIL family member and a pharmaceutically acceptable carrier. As discussed above, a patient can be, without limitation, a human, a non-human primate, a laboratory animals (e.g., mice, rats, rabbits, hamsters, frogs, and fish), a livestock animal (e.g., horses, sheep, cows, pigs, goats, ostriches, chickens, turkeys, ducks, geese, elephants, and llamas), or a pet (e.g., cats and dogs). In some embodiments, the cancerous B cells of the B cell cancer are growth-retarded. In some embodiments, the growth of the cancerous B cells of the B cell cancer is abrogated. In some embodiments, the cancerous B cells of the B cell cancer are killed.

Compositions of FRIL family members may be used safely and efficaciously as therapeutics. For example, the gastrointestinal tracts of animals come in constant contact with lectins, such as FRIL family members, in raw and/or cooked vegetables and fruits. Indeed, many lectins pass through the gastrointestinal tract biologically intact (Pusztai, A., Eur. J. Clin. Nutr. 47: 691-699, 1993). Moreover, some lectins interact with the gut and are transported into the peripheral blood circulation. For example, peanut agglutinin (PNA) was found in the blood of humans at levels of 1-5 μg/ml an hour after they ingested 200 grams of raw peanuts (Wang et al., Lancet 352: 1831-1832, 1998). Antibodies to dietary lectins are commonly found in people at levels of ˜1 μg/ml (Tchemychev and Wilchek, FEBS Lett. 397: 139-142, 1996); however, these circulating antibodies do not block carbohydrate binding of the lectins.

As used herein, the term “therapeutically effective amount” means the total amount of each active component (e.g., a FRIL family member molecule) of a composition that is sufficient to show a meaningful patient benefit. When administered to an animal having a solid tumor, a therapeutically effective amount of the composition of the invention is an amount sufficient to slow tumor growth, or, to arrest tumor growth, or, to diminish tumor size. Such determinations can be made using art-known techniques (e.g., measuring tumor size with calipers, with magnetic resonance imaging (MRI), or with X-ray). When administered to an animal having a non-solid tumor, the cancerous cells may be counted, and a therapeutically effective amount of the composition of the invention will slow the increase in number of cancerous cells, or will prevent an increase in the number of cancerous cells, or will reduce the number of cancerous cells. Such determinations of cell numbers can be made using art-known techniques (e.g., counting cancerous cells with a hemacytometer, a Guava PCA apparatus (see Examples below), or flow cytometer).

In a non-limiting example, when administered systemically, a therapeutically effective amount may be in an amount of between about 500 ng of the FRIL family member/kg total body weight and about 5 mg/kg total body weight per day. In another embodiment, a therapeutically effective amount is between about 500 ng/kg and 500 μg/kg total body weight of the FRIL family member per day, or between about 5 μg/kg and 50 μg/kg total body weight of the FRIL family member per day. In some embodiments, a therapeutically effective amount is an amount that delivers about 50 μg/kg total body weight of the FRIL family member per day.

Administration of a FRIL family member may be with a pharmaceutically acceptable carrier including, without limitation, water, buffered saline, polyol (e.g., glycerol, propylene glycol, liquid polyethylene glycol), or suitable mixtures thereof. Methods for making pharmaceutically acceptable carriers and formulations thereof are found, for example, in Remington's Pharmaceutical Sciences (18th edition), ed. A. Gennaro (1990) Mack Publishing Company, Easton, Pa.

The pharmaceutical formulations and/or compositions of the invention may be administered by any appropriate means. For example, the pharmaceutical formulations and/or compositions of the invention may be administered to a mammal within a pharmaceutically-acceptable diluent, carrier, or excipient, in unit dosage form according to conventional pharmaceutical practice. Formulations for parenteral administration may, for example, contain sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-poloxypropylene copolymers may be used to control the release of the FRIL family member molecule. Other potentially useful parenteral delivery systems include ethylene-vinyl acetate copolymer particles, osmotic pumps, implantable infusion system, and liposomes.

Any appropriate route of administration of a FRIL family member of the invention (or a pharmaceutical formulation and/or composition thereof) may be employed, including, without limitation, parenteral, intravenous, intra-arterial, subcutaneous, sublingual, transdermal, topical, intrapulmonary, intramuscular, intraperitoneal, by inhalation, intranasal, aerosol, intrarectal, intravaginal, or by oral administration. Pharmaceutical formulations and/or compositions of the invention may be in the form of liquid solutions or suspensions; for oral administration, formulations may be in the form of tablets or capsules; and for intranasal formulations, in the form of powders, nasal drops, or aerosols. The pharmaceutical formulations and/or compositions may be administered locally to the area (e.g., directly into the tumor mass), or may be administered systemically. Since the FRIL family member molecules of the invention kill cancerous B cells, but do not kill normal cells, the compositions of the invention may be administered systemically in situations where, for example, the cancer has metastasized throughout the body.

Administration of a FRIL family member molecule may begin before the patient is symptomatic. For example, a patient diagnosed with a B cell cancer (e.g., by virtue of a positive biopsy) who has yet to exhibit characteristic symptoms of cancer (e.g., fatigue, rapidly growing lymph nodes, shortness of breath, pain) may be administered a FRIL family member molecule in accordance with the methods described herein.

Moreover, if desired, treatment of a B cell cancer patient with a FRIL family member molecule of the invention may be combined with more traditional therapies such as surgery, steroid therapy, radiation therapy, chemotherapy, or a combination of one or more of these therapies. Thus, in a further aspect, the invention provides a composition comprising a FRIL family member molecule and a chemotherapeutic, a radiotherapeutic, a steroid, or an agent that selectively kills B cells. Such a composition is useful for killing and/or retarding the growth of cancerous B cells. In some embodiments, the composition further comprises a pharmaceutically acceptable carrier. Non-limiting pharmaceutically acceptable carriers are described above. In some embodiments, the composition is administered to a patient suffering from a B cell cancer.

Non-limiting chemotherapeutics that can be used in the invention include cytarabine, cyclophosphamide (Cytoxan or Endoxana), cytosine arabinoside, doxorubicin (Dox), daunorubicin, 5-fluorouracil (5-FU), alemtuzumab (sold under the name Campath), bexarotene (sold under the name Targretin (LGD 1069)), denileukin diftitox (sold under the name Ontak), Chlorambucil (Leukeran), Fludarabine (Fludara), Cladribine (Leustat), gemtuzumaab-ozogamicin (sold under the name Mylotarg), ibritumomab tiuxetan (sold under the name Zevalin), pegaspargase (sold under the name Oncaspar), rituximab (sold under the name Rituxan), vincristine (Oncovin), prednisolone, etoposide, fludarabine, mitoxantrone, and tretinoin ATRA (sold under the name Vesanoid).

In one non-limiting embodiment of the invention, a composition comprising a FRIL family member molecule and an agent that selectively kills B cells is employed to kill all B cells in a patient suffering from B cell cancer, regardless of whether the B cells killed are cancerous. The normal patient (e.g., a human patient) can generate new, healthy B cell from their bone marrow (or other hematopoietic organ, such as the bursa or fetal liver), and so complete destruction of all B cells will ensure that all cancerous B cells will be killed. As used herein, an “agent that selectively kills B cells” is an agent that preferentially kills B cells but does not kill substantial numbers of non-B cells. In one non-limiting example, such an agent may be an antibody that binds to a cell surface marker that is expressed only on B cells (including, without limitation, CD19, CD20, CD22, CD72, CD79α, CD79β, CD121b, and CD138). Binding of a B cell by the antibody may induce antibody dependent cellular cytotoxicity.

In some embodiments, the antibody may be operably linked to a toxin, such as ricin. In some embodiments, the antibody may be oeprably linked to a radioisotope, such as Yitrium⁹⁰. The term “operably linked” includes any association between the antibody and the agent to which the antibody is linked (e.g., toxin or radioisotope) which allows the antibody to being to a B cell surface marker and allows the agent (e.g., toxin or radioisope) to kill the cell contacted by the antibody.

In a further aspect, the invention features a method for determining if a patient (e.g., a human patient) suffering from a B cell cancer will benefit (i.e., show an alleviation in symptoms) from treatment with a FRIL family member molecule. This method includes contacting a cell from the B cell cancer with a FRIL family member molecule and determining the growth rate of the cell, wherein retardation of growth of the cell contacted with the FRIL family member molecule as compared to a cell from the B cell cancer not contacted with the FRIL family member molecule indicates that patient will benefit from treatment with the FRIL family member molecule. In some embodiments, the patient is then treated by administration of the FRIL family member molecule.

Thus, in particular embodiments, the treatment of a patient suffering from a B cell cancer may take place in two parts. First, a biopsy containing cancerous B cells is taken from an affected area of the patient (e.g., from blood, lymph node, or other tissue). The cancerous B cells from the biopsy are then contacted with a FRIL family member molecule. If the cancerous B cells are sensitive to the FRIL family member molecule (i.e., if contact with a FRIL family member molecule causes retardation of the growth of and/or the death of the cancerous B cells), then the patient can be treated by administering a therapeutically effective amount of a composition that includes a FRIL family member molecule and a pharmaceutically acceptable carrier.

In some embodiments of all of the aspects of the invention, the FRIL family member molecule used in the methods described herein is purified. By “purified” means a molecule, such as a protein (e.g., a FRIL family member molecule or a binding agent or antibody) or composition of that molecule, that is more free from other organic molecules (e.g., carbohydrates, nucleic acids, proteins, and lipids) that naturally occur with an impure molecule, and is substantially free as well of materials used during the purification process. For example, a protein is considered to be purified if it is at least approximately 60%, or at least approximately 75%, or approximately at least 85%, or approximately at least 90%, or approximately at least 95% pure, i.e., 95% free from other organic molecules with which it naturally occurs and 95% free from materials used during the purification process.

FRIL family member molecules are readily purified using standard techniques (including the one described below in the Examples). Additional methods for purifying proteins are known in the art and include, without limitation, HPLC, SDS-PAGE, immunoprecipitation, recombinant protein production, affinity chromatography using specific antibodies, ion-exchange, size-exclusion, and hydrophobic interaction chromatography, or a combination of any of these methods. These and other suitable methods are described, e.g., in Marston, “The purification of eukaryotic proteins expressed in E. coli,” in DNA Cloning, Glover D. M., ed., Volume III, IRL Press Ltd., Oxford, 1987; Marston and Hartley, “Solubilization of protein aggregates,” pp. 266-267 in Guide to Protein Purification, Deutscher M. P., ed., Academic Press, San Diego, 1990; Laemmli, U. K., Nature 227: 680-685, 1970; Ausubel et al., Current Protocols in Molecular Biology, John Wiley & Sons, New York, N.Y., 1999 (updated periodically); U.S. Pat. No. 6,084,060; and Gowda et al., J. Biol. Chem. 269: 18789-18793, 1994. A FRIL family member molecule can also be purified by binding to a mannose, which may be coupled on a sold support (e.g., a sepharose bead). Non-limiting sources from which naturally occurring FRIL family member molecules can be purified include Dolichos lab lab, Phaseolus vulgaris, Sphenostylis stenocarpa, Vigna sinensis, and Voandzeia subterranea.

Purification of a FRIL family member molecule from a legume is rapid and inexpensive, and results in a large amount of purified FRIL family molecule. FRIL family members are relatively abundant in legumes. For example, Dl-FRIL accounts for approximately 0.02% of the mass of hyacinth beans. A FRIL family member molecule can be easily purified from legumes, such as hyacinth beans (which can be grown pesticide-free), by mannose-affinity chromatography, or by ovalbumin affinity chromatography.

Purified FRIL family member molecules may also be made by recombinant methods. A FRIL family member can be synthesized by introducing a nucleic acid sequence encoding the FRIL family member into any appropriate cell type including bacteria, insect, or mammalian, by recombinant techniques well known to those with skill in the art. For example, a FRIL family member-encoding nucleic acid sequence can be inserted into a baculovirus vector such that it is expressed (i.e., transcribed and/or translated) by a cell introduced with the vector. The vector may then be used to generate recombinant baculovirus particles. Insect cells (e.g., Sf9 cells) transduced with the recombinant baculovirus will express the FRIL family member. Following lysis, the FRIL family member can be purified.

In another non-limiting example, recombinant FRIL family member molecules can be produced in dicotyledonous plants, such as Nicotiana tabacus or Arabidopsis thaliana. For example, Arabidopsis plants can be transformed using a strain of Agrobacterium tumefacien carrying a nucleic acid molecule encoding a FRIL family member molecule. Agrobacterium tumefacien infects Arabidopsis by transferring some of its DNA (T-DNA) to the infected Arabidopsis plant. Methods for making vectors for transforming Agrobacterium with a desired nucleic acid molecule are known (see, e.g., McBride and Summerfelt, Plant Mol. Biol. 14(2):269-276, 1990; U.S. Pat. No. 4,940,838 and U.S. Pat. No. 5,464,763). The Agrobacterium strain carrying a nucleic acid molecule encoding a FRIL family member molecule positioned for expression (i.e., positioned such that it will be expressed in an Agrobacterium-transformed plant) can be used to transform Arabidopsis plants, according to standard methods (see, e.g., Ausubel et al., supra), and the recombinant FRIL family member purified from the transformed Arabidopsis plant.

Nucleic sequences encoding FRIL family member molecules can also be expressed in other cell types including, without limitation, CHO cells, plant cells (e.g., Arabidopsis, Lemna, tobacco, or corn), yeast cells, or bacterial cells. Methods for genetically manipulating cells to produced recombinant proteins are well known (see, e.g., Ausubel et al., supra).

In yet another aspect, the invention provides a method for locating a cancerous B cell in a patient (e.g., a human patient) comprising administering a detectably labeled FRIL family member molecule to the patient and detecting the label. In accordance with this aspect of the invention, the label will be located at a position in the body where there are cancerous B cells. These areas (e.g., lymph nodes) of the body can be excised to remove the cancerous B cells.

In a further non-limiting example, this method of the invention can be used following treatment to remove cancerous B cells. For example, a patient suffering from a B cell cancer (e.g., non-Hodgkin's lymphoma) can be treated with a FRIL family member, and/or with more conventional therapies (e.g., surgery, chemotherapy, and/or radiotherapy) to remove cancerous B cells. Following treatment, a detectably labeled FRIL family member can be administered to the patient to determine if any cancerous B cells remain in the patient.

As used herein, by “detectably labeled” is meant that the FRIL family member molecule is attached to a label that is detectable visually or instrumentally. For example, a chromophoric or fluorogenic molecule can be conjugated to the FRIL family member molecules or binding agent by means of coupling agents, such as dialdehydes, carbodiimides, and dimaleimides. Non-limiting detectable labels include biotin, phycoerythrin, and FITC. The FRIL family member molecule may also be detectably labeled by being attached to a radiolabel (e.g., ³H, ³²P, or ³⁵S), such that it can be detected with an X-ray machine. Accordingly, when population of cells is contacted with a detectably labeled FRIL family member molecule (or with the FRIL family member molecule followed by a detectably labeled binding agent, such as an antibody, that specifically binds to the FRIL family member molecule), bound cells can be isolated by cell sorting on a flow cytometry instrument.

In one non-limiting example of this aspect of the invention, a patient with metastatic lymphoma may be systemically administered a detectably labeled FRIL family member molecule (e.g., with a radiolabeled FRIL family member molecule), and then exposed to an X-ray to determine the location of the cancerous B cells. The patient's lymph nodes bearing the label (and so bearing cancerous B cells) can then be excised.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLE I

### FRIL Binds to Human B cell Malignancies with High Affinity

The affinity of a non-limiting FRIL family member molecule, namely Dl-FRIL, to different malignant B cell lines and normal B cells was determined.

For these studies, normal human B cells were isolated from peripheral blood of healthy normal volunteers using the Rosette-Sep B-cell separation antibody cocktail (commercially available from StemCell Technologies) to remove contaminating non-B white and red blood cells. The purity of the preparations was determined fluorometrically using an anti-CD19 antibody conjugated to phycoerythrin (BD Pharmingen) and then analyzing the cells using a Guava PCA (Guava Technologies, Inc., Hayward, Calif.). B cell preparations were >70% CD19-positive.

In addition, various cancerous B cell lines, namely CCRF-SB (a Burkitt's B-ALL cell line) and JM-1 (a Non-Hodgkin's Lymphoma cell line), were obtained from the American Type Culture Collection (Manassas, Va.) and cultured in RPMI complete (with 10% fetal bovine serum, 50 μM 2-mercaptoethanol, and gentamicin) media.

The DI-FRIL employed in these studies was isolated from the Hyacinth bean, Dolichos lablab, as previously described (see, e.g., Colucci et al., Proc. Natl. Acad. Sci. USA 96: 646-650, 1999). Briefly, seeds from the hyacinth beans (Dolichos lab lab) were purchased from Stokes Seeds (Buffalo, N.Y.) and grown in a greenhouse. Dry seeds were ground in a coffee mill and the powder was extracted in 5 volumes of 50 mM Tris/HCl containing 1 nM each of MgCl₂ and CaCl₂ for 4 hours at 4° C. Bean solids were pelleted by centrifugation at 10,000×g for 20 minutes. The pH of the supernatant was acidified to pH 4.0 with acetic acid, followed by a second centrifugation to clarify the supernatant, and finally the pH was readjusted to 8.0 with sodium hydroxide. This crude extract was stored at −20° C. Single-step purification of the FRIL family member was achieved by binding to a methyl α-D-mannopyanoside Sepharose matrix (Sigma). The gel (i.e., matrix) was tumbled with the thawed crude extract for 10 minutes at 22° C., carefully washed several times with 50 mM Tris/HCl containing 1 nM each of MgCl₂ and CaCl₂, and then eluted with 100 mM mannose (commercially available from Sigma Chemical Co., St. Louis Mo.). Because this FRIL family member was isolated from Dolichos lab lab, it is referred to herein as Dl-FRIL. This purified Dl-FRIL was greater than 98% Dl-FRIL as assessed by HPLC.

Dl-FRIL was biotinylated to produce biotinylated Dl-FRIL (Dl-FRIL-Bi) by incubating 2 mg of Dl-FRIL with 20-fold molar excess of sulfo-NHS-biotin (commercially available from Sigma Chemical Co.) for 30 minutes at room temperature as described in the manufacturer's instructions.

5×10⁵ cancerous or normal B-cells were harvested, washed in saline solution, and incubated with different amounts of biotinylated Dl-FRIL (i.e., 0.1, 0.5, 1, 2.5, 10, and 25 μg/ml for 15 minutes at 4° C. The cells were then washed and incubated with 0.1 μg streptavidin-PE (SA-PE; commercially available from Southern Biotech, Birmingham, Ala.) for 10 minutes at 4 C. The cells were then washed and analyzed for FRIL binding fluorometrically using Guava PCA (Guava Technologies, Inc.).

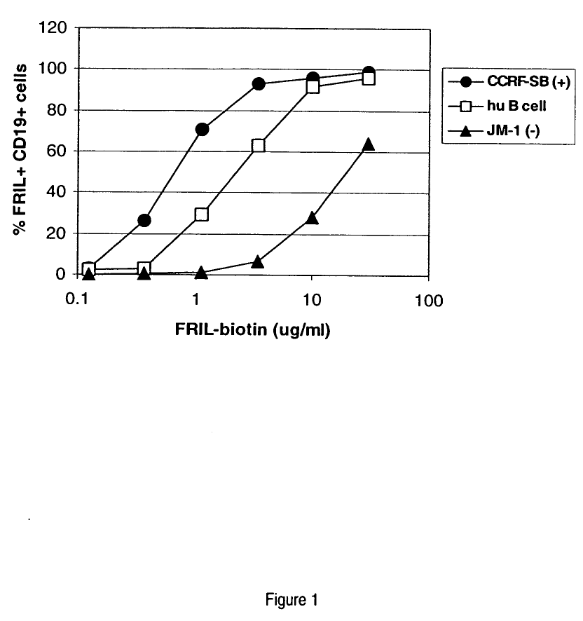

As shown in FIG. 1, biotinylated Dl-FRIL bound with higher affinity to one of the tumor cell lines, namely CCRF-SB, than to normal human B cells. Interestingly, biotinylated Dl-FRIL did not bind as well to the JM-1 tumor cell line. As normal human B cells, CCRF-SB cells, and JM-1 cells do not express the FLT3 receptor, the ligand on these cells to which the Dl-FRIL binds must be something other than the FLT3 receptor.

Moreover, Dl-FRIL's binding to normal human B cells, the JM-1 cells, and the CCRF-SB cells did not appear to induce resting nor did it impede their activation with various agonists. Interestingly, the CCRF-SB cells (which bound to the Dl-FRIL with the highest affinity) appeared to be killed by the binding. In experiments where B cells were activated with anti-IgM and IL-4 or LPS, the normal induction of the activation marker B7.2 (CD86) was observed with some loss of activated B cells (data not shown).

### EXAMPLE II

### FRIL Kills the Cancerous B cells to Which it Binds with High Affinity

Based on the observation in Example I that the CCRF-SB cells, which bound to Dl-FRIL with highest affinity, were killed by the binding to Dl-FRIL, the binding of Dl-FRIL to different cancerous B cells was tested. For these studies, Raji cells (a Burkitt's lymphoma line), SR (a leukemia cell line), HT (a diffuse mixed lymphoma cell line) and two additional B cell Non-Hodgkin's Lymphoma lines, MC116 and RL, were obtained from the American Type Culture Collection (ATCC; Manassas, Va.) and cultured in RPMI complete (with 10% fetal bovine serum, 50 μM 2-mercaptoethanol, and gentamicin) media. KG1a cells (a myeloid lymphoma cell line) were also obtained from a colleague and cultured in RPMI complete (with 10% fetal bovine serum, 50 μM 2-mercaptoethanol, and gentamicin) media.

These tumor lines, as well as the CCRF-SB and JM1 cells (described in Example I) were subjected to staining with biotinylated Dl-FRIL as described in Example I. Staining with Dl-FRIL revealed that the Raji, HT, CCRF, and MC116 cell lines were bound by DI-FRIL with high affinity. RL cells bound FRIL at intermediate concentrations. The KG1a, SR, and JM1 cell lines did not bind DI-FRIL (data not shown).

Next, all of the tumor lines were subjected to a killing assay with Dl-FRIL, to determine if Dl-FRIL, a non-limiting FRIL family member of the invention, was able to kill these cells. For this killing assay, tumor cells were incubated for 48 hours at 37° C. in RPMI complete media with 0.312, 0.625, 1.25, 2.5, 5, 10, and 20 μg/ml of purified FRIL (i.e., non-biotinylated FRIL). Viable cell counts were then determined by adding XTT to cultures. XTT can be metabolically reduced by mitochondrial dehydrogenase in viable cells to a water-soluble highly colored formazan product, which can be measured. The cells were incubated for 2 hours at 37° C. with XTT, and then the absorbance at read at 450 nm-650 nm to determine metabolic activity.

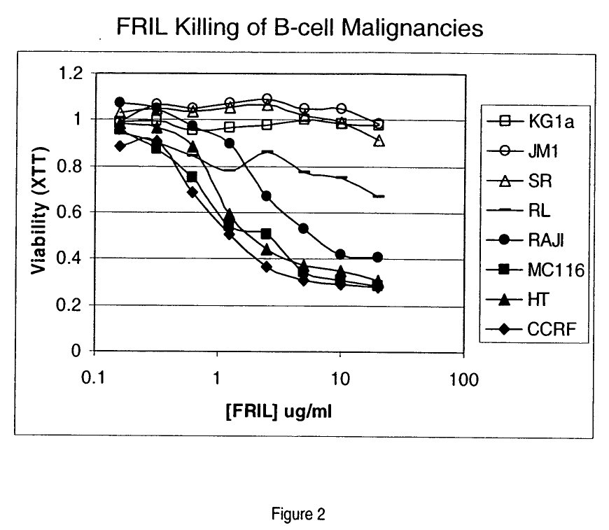

As shown in FIG. 2, those cells that bound to biotinylated Dl-FRIL with high affinity (namely Raji, HT, CCRF, and MC116) were killed by purified Dl-FRIL. RL cells, which FRIL bound with intermediate affinity demonstrated moderate killing by FRIL. Normal B cells are predicted to bind FRIL with a slightly lower affinity than RL cells, and are probably killed at a level slightly lower than RL cells by FRIL.

The three cell lines that did not bind biotinylated DI-FRIL with high affinity were not killed by purified Dl-FRIL. Not surprisingly, KG1a cells (open squares) and SR cells (open triangles), which are not cancerous B cells, were two of the three cell lines which were not bound with high affinity (or killed) by DI-FRIL. Moreover, although the third cell line not killed by Dl-FRIL, namely the JM1 cells, are cancerous B cells, it is possible that because this cell line lost expression of the cell surface molecule to which DI-FRIL binds during its propagation in vitro. Another possibility is that because JM1 cells are pre-B cells, it may be that they do not express as high levels of the FRIL ligand as the more mature cancerous B cells.

Next, the killing assay was repeated, with viability cell counts being assessed by determining the percentage of cells negative for uptake of 7-AAD (a cell impermeable dye) and annexin-V (which binds to inverted phosphatidyl serine on apoptotic cells) using the Guava PCA (Guava Technologies, Inc.). The results are shown in Table II.

TABLE II

| | | | | Killed |
|---|---|---|---|---|
| | | Cell | | by |
| Cell line | Classification | type | Isotype | FRIL? |
| MC116 | Undifferentiated | B cell | IgM | Yes |
| | lymphoma | | | |
| CCRF-SB | Acute lymphocytic leukemia | B cell | — | Yes |
| RAJI | Burkitt's lymphoma | B cell | IgM | Yes |
| RL | Non-Hodgkin's lymphoma | B cell | IgM | Yes |
| HT | Diffuse mixed lymphoma | B cell | IgM | Yes |
| JM1 | Immunoblastic lymphoma- | Pre-B | ?? | No |
| | leukemia | cell | | |
| KG1a | Acute myelogenous leukemia | Myeloid | ?? | No |
| SR | Lymphoblastic leukemia | ?? | ?? | No |
| Human | Normal | B cell | IgM, IgG | Low |
| B cells | | | | |

As shown in Table II (based on the use of FRIL at different concentrations), the only cancerous B cell line not killed by contact with Dl-FRIL was the JM-1 cells, while both the non-B cell lines, KG1a and SR, were not killed by contact with Dl-FRIL. The remaining cancerous B cell line lines, namely MC116, CCRF-SB, Raji, RL, and HT were killed by contact with Dl-FRIL.

These results showed that high affinity binding of various cancerous B cells to purified Dl-FRIL (a non-limiting FRIL family member) causes a dramatic killing of these cells (FIG. 2). Within several hours of contact with FRIL, B-cell tumor necrosis was observed. Moreover, this killing did not require the presence of immune complement. Killing of B cell derived tumor cells was specific as FRIL did not kill tumor derived cell lines from other hematopoietic malignancies, including T cell and myeloid leukemias, nor did FRIL kill breast, lung, colon or brain tumor derived cell lines (data not shown).

### EXAMPLE III

### FRIL Kills the Cancerous B Cells with High Efficiency

The kinetics of killing by FRIL was next determined. Normal peripheral B cells and MC116 cells (a FRIL-sensitive B cell non-Hodgkin's lymphoma cell line) were cultured in the presence of 0.2 μg/ml or 1 μg/ml purified Dl-FRIL. At various times following contact (i.e., 30 minutes, 60 minutes, 180 minutes, and 360 minutes following contact), viable cells were counted as described above in Example II.

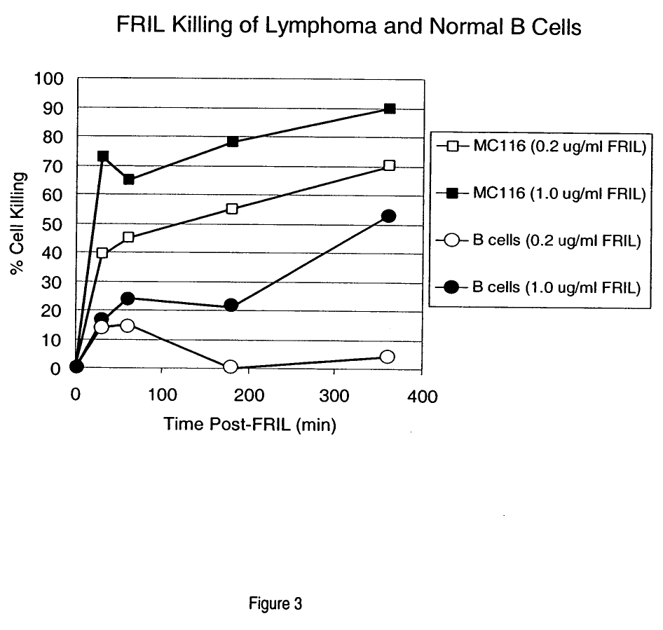

As shown in FIG. 3, within 30 minutes of contact with FRIL, cancerous B cell death was observed even at sub-microgram/ml concentrations of FRIL (see FIG. 3, white squares). Within 6 hours, over 90% of the lymphoma cells in the cultures containing 1.0 μg/ml purified Dl-FRIL were killed (FIG. 3, black squares) and longer term cultures did not demonstrate viable cells growing out of these cultures (data not shown). This killing was complement independent, and was not mediated through an antibody dependent cell cytotoxicity (ADCC) mechanism. Moreover, this killing did not require a radio-isotope. Low levels of killing of normal human B cells was also observed at sub-microgram/ml concentrations of FRIL (FIG. 3, white circles) with higher levels of normal B cell killing observed at higher concentrations of FRIL over time (FIG. 3, black circles). Thus, FRIL demonstrates a significant therapeutic ratio.

### EXAMPLE IV

Killing of Cancerous B Cells by Different FRIL Family Members

Equivalency of killing of lympohoma cells by several members of the FRIL family was next determined. CCRF cells were cultured for 48 hours at 37° C. in RPMI complete media with 0.78, 0.156, 0.312, 0.625, 1.25, 2.5, 5, and 10 μg/ml purified Dl-FRIL or PvFRIL. Viable cells were then counted as described above in Example II.

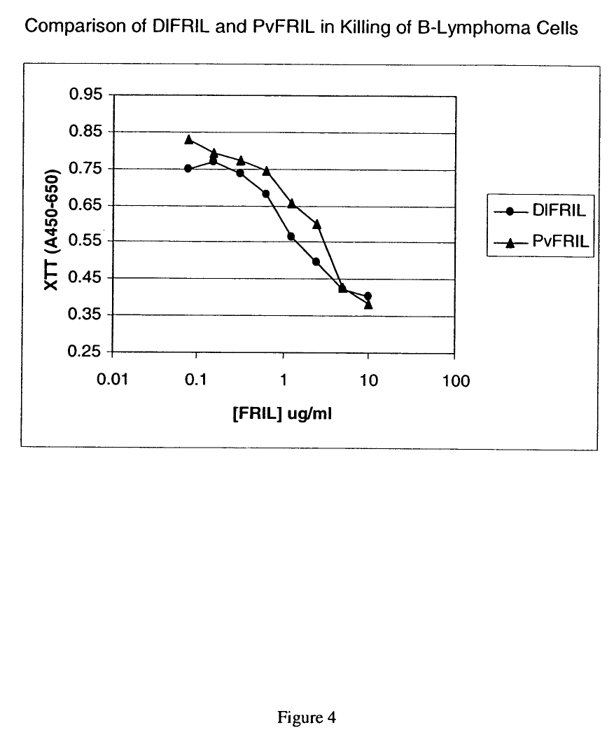

The results show that the cancerous B cells are killed with approximately efficiencies by the different FRIL family members (FIG. 4). In contrast, minimal B lymphoma cell killing was observed in the plates containing no lectin (data not shown).

### EXAMPLE V

### FRIL Specifically Kills Cancerous B Cells

To determine whether the killing activity of a FRIL family member is specific to cancerous B cells, mixed cultures of normal B cells and a cancerous B cell line sensitive to FRIL (e.g., HT) are contacted with a FRIL family member molecule by adding a purified FRIL family member molecule to the culture media. After a specified amount of culture time, the remaining cells are analyzed.

For this study, HT cells from the ATCC are cultured in RPMI complete (with 10% fetal bovine serum, 50 μM 2-mercaptoethanol, and gentamicin) media. HT cells are derived from a diffuse mixed lymphoma of a white Caucasian male human.

Next, normal human B cells are isolated from peripheral blood of healthy normal female volunteers using the Rosette-Sep B-cell separation antibody cocktail (commercially available from StemCell Technologies) to remove contaminating non-B white and red blood cells. The purity of the preparations is determined fluorometrically by staining the cells with an anti-CD19 antibody conjugated to phycoerythrin (BD Pharmingen) and then analyzing the cells using a Guava PCA (Guava Technologies, Inc., Hayward, Calif.). B cell preparations are >70% CD19-positive.

The normal human B cells are mixed together with the HT cells and cultured at 37° C. in the presence of 0.2 μg/nm or 1.0 μg/nm of a purified FRIL family member molecule (e.g., Dl-FRIL). Control plates containing no FRIL, and plates containing only B cells or only HT cells are also set up. Viable cells are counted daily. In addition, DNA or RNA from the viable cells are isolated and subjected to PCR analysis to determine if the cells contain the Y chromosome. Only HT cells will contain the Y chromosome since the normal B cells are from female donors.

The results will show that the viable cells from the cultures all lack the Y chromosome, meaning that the viable cells are all from the normal human B cells. These results will show that contact with FRIL kills cancerous B cells, but leaves normal B cells unscathed.

### EXAMPLE VI

FRIL Kills Cancerous B Cells without Harming Progenitor Cells

In a study similar to that described in Example V, HT cells are cultured together with umbilical cord blood cells from a female human. The cultured cells are contacted with a FRIL family member molecule by adding the FRIL family member molecule to the culture medium. After culture for a specified period of time (e.g., a week), the viable cells counted and their DNA analyzed to determine if they contain the Y chromosome.

The results will show that after contact with the FRIL family member molecule, the only viable cells remaining in the culture are female cells (i.e., lacking the Y chromosome). These cells are progenitor cells and normal B cells derived from the female umbilical cord blood. The viable cells, although put to “sleep” by the FRIL family member, are alive, and will “wake up” once the FRIL family member molecule is depleted from the culture media. Their “awakening” (i.e., return to normal rate of replication) can be hastened by rinsing the old culture media off the cells and replating them in media lacking the FRIL family member molecule.

### EXAMPLE VII

### Treatment of a Human Suffering from B Cell Non-Hodgkin's Lymphoma with FRIL

A biopsy containing cancerous B cells is taken from a human subject suspected of suffering from B cell Non-Hodgkin's lymphoma. The cells of the biopsy are contacted with a FRIL family member molecule to determine if they are sensitive to the FRIL family member molecule (i.e., if they are growth retarded and/or killed when contacted with the FRIL family member molecule).

If the patient's cancerous B cells are sensitive to the FRIL family member molecule, the patient next receives treatment by administration of a therapeutically effective amount of the FRIL family member molecule. Although the administration can take any route, in this example, the patient receives between about 5 μg/kg and 50 μg/kg total body weight of the FRIL family member in physiological saline solution per day intravenously.

The patient will be found to have benefited (i.e., shows an improved in his lymphoma symptoms) following treatment with the FRIL family member molecule.

### EXAMPLE VIII

### Treatment of a Human Suffering from B cell Acute Lymphocytic Leukemia (ALL) with FRIL

Two patients suffering from ALL are initially treated for the first seven days with daunorubicin at 45 mg/m² on Days 1-3 plus cytarabine at 100 mg/m² for 7 days.

One of the patients receives, in addition to the daunorubicin and cytarabine chemotherapeutics, a therapeutically effective amount of a FRIL family member molecule. The patient receiving the FRIL family member molecule will show a better prognosis for ALL than the patient who received only the chemotherapeutics.

1. A method for killing a cell of a B cell cancer, comprising contacting the cell with a FRIL family member molecule.

2. A method for retarding the growth of a cell of a B cell cancer, comprising contacting the cancerous B cell with a FRIL family member molecule, wherein the FRIL family member molecule binds to a molecule on the surface of the cell that is not a normally glycosylated FLT3 receptor.

3. The method of claim 1 or 2, wherein the cell is cultured in vitro.

4. The method of claim 1 or 2, wherein the cell is in a patient.

5. The method of claim 4, wherein the patient is a human.

6. The method of claim 4, wherein the FRIL family member molecule is administered to the patient.

7. A method for determining if a B cell cancer is sensitive to a FRIL family member molecule comprising contacting a cell from the B cell cancer with the FRIL family member molecule and determining the growth rate of the cell, wherein retardation of growth of the cell contacted with the FRIL family member molecule as compared to a cell from the B cell cancer not contacted with the FRIL family member molecule indicates that the B cell cancer is sensitive to the FRIL family member molecule.

8. A method for determining if a patient suffering from a B cell cancer will benefit from treatment with a FRIL family member molecule, comprising contacting a cell from the B cell cancer with a FRIL family member molecule and determining the growth rate of the cell, wherein retardation of growth of the cell contacted with the FRIL family member molecule as compared to a cell from the B cell cancer not contacted with the FRIL family member molecule indicates that patient will benefit from treatment with the FRIL family member molecule.

9. The method of claim 2, 7, or 8, wherein the growth of the cell is abrogated.

10. The method of claim 2, 7, or 8, wherein the cell is killed.

11. A method for treating a patient suffering from a B cell cancer, comprising administering to the patient a composition comprising a therapeutically effective amount of a FRIL family member and a pharmaceutically acceptable carrier.

12. The method of claim 11, wherein the patient is a human.

13. The method of claim 11, wherein the growth of a cell of the B cell cancer is retarded.

14. The method of claim 11, wherein the growth of a cell of the B cell cancer is abrogated.

15. The method of claim 11, wherein a cell of the B cell cancer is killed.

16. A composition comprising a FRIL family member molecule and an agent selected from the group consisting of a chemotherapeutic, a radiotherapeutic, and an agent that selectively kills B cells.

17. The method of claim 16, wherein the composition further comprises a pharmaceutically acceptable carrier.

18. A method for locating a B cell cancer in a patient comprising administering a detectably labeled FRIL family member molecule to the patient and detecting the label.

19. The method of claim 18, wherein the patient is a human.

20. The method of claim 18, wherein the label is selected from the group consisting of a radiolabel, a chromophoric label, and a fluorophoric label.

21. The method of claim 1, 2, 7, 8, 11, 16, or 18, wherein the FRIL family member molecule is purified.

22. The method of claim 1, 2, 7, 8, 11, 16, or 18, wherein the FRIL family member molecule is selected from the group consisting of Dl-FRIL, Yam-FRIL, and Pv-FRIL.

23. The method of claim 1, 2, 7, 8, 11, 16, or 18, wherein the B cell cancer is a B cell Non-Hodgkin Lymphoma.

24. The method of claim 23, wherein the B cell Non-Hodgkin Lymphoma is selected from the group consisting of a small lymphocytic lymphoma (SLL), a mantle cell lymphoma, a Burkitt's lymphoma, a follicle centre cell lymphoma, a follicular lymphoma, a Burkitt-like lymphoma, a marginal zone B-cell lymphoma (MZBCL), a nodal marginal zone B cell lymphoma, an extra-nodal marginal zone B cell lymphoma, a splenic marginal zone B cell lymphoma, a lymphoplasmacytic lymphoma, and a diffuse large B cell lymphoma.

25. The method of claim 1, 2, 7, 8, 11, 16, or 18, wherein the B cell cancer is selected from the group consisting of a B cell acute lymphocytic leukemia (B-ALL), a precursor B cell acute lymphocytic leukemia (B-ALL), a B cell chronic lymphocytic leukemia (B-CLL), a precursor B-lymphoblastic leukaemia, a precursor B-lymphoblastic lymphoma, a small lymphocytic lymphoma, a B cell prolymphocytic leukemia, an undifferentiated B cell lymphoma, a hairy cell leukemia, a mediastinal large B-cell lymphoma, a plasma cell myeloma, a plasmacytoma, a primary effusive lymphoma, a Burkitt's cell leukemia, and a B cell diffuse mixed lymphoma.


SEQUENCE LISTING (6 sequences)

SEQ ID NO: 1 - DNA, 861 bp, Lablab purpureus
atggcttcctccaacttactcaccctagccctcttccttgtgcttctcacccacgcaaactcagccgcacagtcattgtcatttagtttcaccaagtttgatcctaaccaagaggatcttatcttccaaggtcatgccacttctacaaacaatgtcttacaactcaccaagttagacagtgcaggaaaccctgtgagttctagtgcgggaagagtgttatattctgcaccattgcgcctttgggaagactctgcggtattgacaagctttgacaccattatcaactttgaaatctcaacaccttacacttctcgtatagctgatggcttggccttcttcattgcaccacctgactctgtcatcagttatcatggtggttttcttggactctttcccaacgcaaacactctcaacaactcttccacctctgaaaaccaaaccaccactaaggctgcatcaagcaacgttgttgctgttgaatttgacacctatcttaatcccgattatggtgatccaaactacatacacatcggaattgacgtcaactctattagatccaaggtaactgctaagtgggactggcaaaatgggaaaatagccactgcacacattagctataactctgtctctaaaagactatctgttactacttattatcctgggagtaaacctgcgactctctcctatgatattgagttacatacagtgcttcctgaatgggtcagagtagggttatctgcttcaactggacaagataaagaaagaaataccgttcactcatggtctttcacttcaagcttgtggaccaatgtggcgaagaaggagaatgaaaacaagtatattacaagaggcgttctgtga

SEQ ID NO: 2 - protein, 286 aa, Lablab purpureus
MASSNLLTLALFLVLLTHANSAAQSLSFSFTKFDPNQEDLIFQGHATSTNNVLQLTKLDSAGNPVSSSAGRVLYSAPLRLWEDSAVLTSFDTIINFEISTPYTSRIADGLAFFIAPPDSVISYHGGFLGLFPNANTLNNSSTSENQTTTKAASSNVVAVEFDTYLNPDYGDPNYIHIGIDVNSIRSKVTAKWDWQNGKIATAHISYNSVSKRLSVTTYYPGSKPATLSYDIELHTVLPEWVRVGLSASTGQDKERNTVHSWSFTSSLWTNVAKKENENKYITRGVL

SEQ ID NO: 3 - protein, 276 aa, Lablab purpureus
MFPSKVKSAQSLSFSFTKFDPNQKDLIFQGDVTSTNNVLQLTKLDSAGKPLGSSVGRVLYSAPLHLWEDSAVLSSFDTTITFEISTPYTSHAADGFTFFLAPLDTVIPVNSFGRLLGLFPNTNTFKNSSTSENQTTIDFKAASNSVVAVEFDTYPNVDFGDPNYTHIGIDVNSIKSKVTAMWDWQNGKTATAHISYNSVSKRLSVTTYYPGSKPATLSYDIELHTVLPEWVRVGLSASTGEDKERNTVHSWSFTSSLWNNVAKKEKEDKYITRGVL

SEQ ID NO: 4 - protein, 237 aa, Lablab purpureus
AQSLSFSFTKFDPNQEDLIFQGTATSKLDSAGNPVSSSAGRVLYSAPLRLWEDSAVLTSFDPTIYIFTNYTSRIADGLAFIAPPDSVISYHGGFLGLFPNAAESGIAESNVVAVEFDTDYLNPDYGDPNYIHIGIDVNSIRSKVTASWDWQNGKIATAHISYNSVSKRLSVTTYYPGRGKPATSYDIELHTVLPEWVRVGLSASTGQNIERNTVHSWSFTSSLWTNVAKVGVASISG

SEQ ID NO: 5 - DNA, 906 bp, Phaseolus vulgaris
atggcttcctccaacttactcaccctagccctcttccttgtgcttctcacccacgcaaactcagccgctcagtcattatcttttaactttaccaagtttgatcttgaccaaaaagatcttatcctccaaggtgatgccacttctacaaacaatgtcttacaactcactaagttagacagtggaggaaaccctgtgggtgctagtgtgggaagagtgttattctctgcaccatttcatctttgggaaaactctatggcagtgtcaagctttgaaactaatctcaccattcaaatctcaacacctcacccttattatgcagctgatggctttgccttcttccttgcaccacatgacactgtcatccctccaaattcttggggcaaattccttggactctactcaaacgttttcagaaactcccccacctctgaaaaccaaagctttggtgatgtcaatactgactcaagagttgttgctgtcgaatttgacaccttccctaatgccaatattgatccaaattacagacacattggaatcgatgtgaactctattaagtccaaggaaactgctaggtgggagtggcaaaatgggaaaacggccactgcacgcatcagctataactctgcctctaaaaaactaactgttactacgttttatcctgggatggaagttgtggctctctcccatgatgttgacttacatgcagagcttcctgaatgggttagagtagggttatctgcttcaactggagaggagaaacaaaaaaataccattatctcatggtctttcacttcaagcttgaagaacaacgaggtgaaggagccgaaagaagacatgtatattgcaaacgttgtgcgatcatatacatggatcaatgacgttctatcttatataagcaataaataa

SEQ ID NO: 6 - protein, 301 aa, Phaseolus vulgaris
MASSNLLTLALFLVLLTHANSAAQSLSFNFTKFDLDQKDLILQGDATSTNNVLQLTKLDSGGNPVGASVGRVLFSAPFHLWENSMAVSSFETNLTIQISTPHPYYAADGFAFFLAPHDTVIPPNSWGKFLGLYSNVFRNSPTSENQSFGDVNTDSRVVAVEFDTFPNANIDPNYRHIGIDVNSIKSKETARWEWQNGKTATARISYNSASKKLTVTTFYPGMEVVALSHDVDLHAELPEWVRVGLSASTGEEKQKNTIISWSFTSSLKNNEVKEPKEDMYIANVVRSYTWINDVLSYISNK